(19) United States
(12) Patent Application Publication
(10) Pub. No.: US 2025/0256073 A1
(43) Pub. Date: Aug. 14, 2025

(54) EXPANDABLE CATHETER

(71) Applicant: MicroVention, Inc., Aliso Viejo, CA (US)

(72) Inventor: Belete Degaga, Aliso Viejo, CA (US)

(73) Assignee: MicroVention, Inc., Aliso Viejo, CA (US)

(21) Appl. No.: 19/048,722

(22) Filed: Feb. 7, 2025

Related U.S. Application Data
(60) Provisional application No. 63/551,363, filed on Feb. 8, 2024.

(51) Int. Cl. A61M 25/10; A61F 2/24; A61F 2/958

(52) U.S. Cl. CPC A61M 25/1011; A61F 2/2427; A61F 2/958; A61M 2025/1013; A61M 2025/1061

(57) ABSTRACT

An expandable catheter system includes a catheter body and a plurality of expansile elements connected at a distal region of the catheter body. The system further comprises an expansion mechanism capable of independently adjusting the radial size of each expansile element when in an actuated state. This independent adjustment allows for precise control over the expansion of each element, facilitating targeted treatment or intervention within a body lumen. The system is designed to enhance the adaptability and effectiveness of catheter-based procedures by providing customizable expansion capabilities tailored to specific anatomical or procedural requirements.

### RELATED APPLICATIONS

This application claims benefit of and priority to U.S. Provisional Application Ser. No. 63/551,363 filed Feb. 8, 2024, entitled Balloon Catheter, which is hereby incorporated herein by reference in its entirety.

### BACKGROUND

Stents, artificial heart valves, and similar implantable devices are often expanded within a patient by inflating a balloon catheter within the device. Similarly, narrowed vessels, such as those narrowed by vascular plaque, are often treated by angioplasty in which a balloon catheter is inflated at the narrowed region.

Balloon catheters typically include an elongated catheter body, a balloon at or near a distal end of the balloon body, and an inflation lumen within catheter body that is in communication with an interior of the balloon. Inflation media may be delivered through the inflation lumen to inflate the balloon during a procedure. As the balloon expands, it radially expands the device and/or vessel to a desired size.

### SUMMARY

In some aspects, the techniques described herein relate to an expandable catheter system, including: a catheter body; a plurality of expansile elements connected at a distal region of the catheter body; and, an expansion mechanism having an actuated state that independently adjusts a radial size of each of the plurality of expansile elements.

In some aspects, the techniques described herein relate to an expandable catheter system, wherein the plurality of expansile elements include balloons and wherein the expansion mechanism includes an inflation tube longitudinally movable within the catheter body between at least a first position in communication with a first balloon of the plurality of balloons, and a second position in communication with a second balloon of the plurality of balloons.

In some aspects, the techniques described herein relate to an expandable catheter system, wherein the catheter body includes an interior body passage extending between a proximal region and a distal region of the catheter body; and wherein the inflation tube is longitudinally movable within the interior body passage between the at least first position and second position.

In some aspects, the techniques described herein relate to an expandable catheter system, wherein of all of the plurality of balloons, the inflation tube is only in communication with the first balloon when in the first position.

In some aspects, the techniques described herein relate to an expandable catheter system, wherein of all of the plurality of balloons, the inflation tube is only in communication with the second balloon when in the second position.

In some aspects, the techniques described herein relate to an expandable catheter system, wherein of all of the plurality of balloons, the inflation tube is only in communication with the first balloon when in the first position and wherein the inflation tube is only in communication with the first balloon and the second balloon when in the second position.

In some aspects, the techniques described herein relate to an expandable catheter system, wherein the first balloon and the second balloon each include inflation chambers that are completely isolated from each other, partially in communication with each other, or selectively in communication with each other.

In some aspects, the techniques described herein relate to an expandable catheter system, wherein the catheter body includes a first catheter body port located underneath the first balloon and a second catheter body port located underneath the second balloon.

In some aspects, the techniques described herein relate to an expandable catheter system, wherein the inflation tube further includes an inflation port opening through a sidewall of the inflation tube and into an inflation passage of the inflation tube.

In some aspects, the techniques described herein relate to an expandable catheter system, further including rotational orientation lock that maintains a rotational orientation of the inflation tube relative to the catheter body.

In some aspects, the techniques described herein relate to an expandable catheter system, further including one or more seals located around 1) the inflation port opening and/or located around 2) the first catheter body port and the second catheter body port.

In some aspects, the techniques described herein relate to an expandable catheter system, further including a stent positioned over the plurality of balloons.

In some aspects, the techniques described herein relate to an expandable catheter system, further including a replacement valve framework positioned over the plurality of balloons.

In some aspects, the techniques described herein relate to an expandable catheter system, wherein at least some of the plurality of balloons have inflated diameters that are different from each other.

In some aspects, the techniques described herein relate to an expandable catheter system, wherein the plurality of balloons includes the first balloon, the second balloon, and a third balloon, and wherein the second balloon is positioned between the first balloon and the third balloon, and wherein an inflated diameter of the second balloon is larger or smaller than inflated diameters of the first balloon and the third balloon.

In some aspects, the techniques described herein relate to an expandable catheter system, wherein the plurality of balloons includes the first balloon, the second balloon, and a third balloon, and wherein the inflation tube is moved between the first position, the second position, and a third position to sequentially inflate the first balloon first, the second balloon second, and the third balloon third.

In some aspects, the techniques described herein relate to an expandable catheter system, wherein the inflation tube is movable to a third position to remove inflation fluid from the plurality of balloons.

In some aspects, the techniques described herein relate to an expandable catheter system, wherein the plurality of expansile elements include a plurality of mesh structures and wherein the expansion mechanism includes one or more control wires connected to the plurality of mesh structures.

In some aspects, the techniques described herein relate to a balloon catheter system, including: a catheter body including an interior body passage extending between a proximal region and a distal region of the catheter body; a plurality of balloons connected at the distal region of the catheter body; wherein each balloon is separately in communication with the interior body passage; and, an inflation tube including a distal inflation opening; wherein the inflation tube is longitudinally movable between at least a first position in communication with a first balloon of the plurality of balloons, and a second position in communication with a second balloon of the plurality of balloons.

In some aspects, the techniques described herein relate to a balloon catheter system, including: a catheter body; a plurality of balloons connected at a distal region of the catheter body; and, an inflation tube means for longitudinally moving between positions within the catheter body and establishing an inflation passage between only one of the plurality of balloons at a time.

In some aspects, the techniques described herein relate to a method of using a balloon catheter system, including: longitudinally aligning a distal inflation opening of an inflation tube with a first port of an interior body passage of a catheter body; inflating a first balloon in communication with the first port; longitudinally aligning the distal inflation opening of the inflation tube with a second port of an interior body passage of the catheter body; and, inflating a second balloon in communication with the second port.

In some aspects, the techniques described herein relate to a method, further including longitudinally aligning the distal inflation opening of the inflation tube with a third port of the interior body passage of the catheter body, and inflating a third balloon in communication with the third port.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates a side view of an example balloon catheter system 100 with a plurality of balloons 104A, 104B, and 104C located within a distal region 102D of a catheter body 102 that may be separately and independently inflated relative to each other, according to some examples.

FIG. 2 illustrates a perspective view of a portion of the distal region 102D of the catheter body 102, according to some examples.

FIG. 3 illustrates a side view of a balloon assembly 104 comprising the plurality of balloons 104A, 104B, and 104C, according to some examples.

FIG. 4 illustrates a side view of the distal region 102D of the catheter body 102 comprising catheter body ports 102A, 102B, and 102C, according to some examples.

FIG. 5 illustrates a side view of the inflation tube 106, according to some examples.

FIG. 6 illustrates balloons 104A, 104B, and 104C in a deflated state, according to some examples.

FIG. 7 illustrates balloon 104C at an inflated state in response to injecting inflation media when the inflation opening 106A is aligned with catheter body port 102C, according to some examples.

FIG. 8 illustrates balloons 104C and 104B in an inflated state, according to some examples.

FIG. 9 illustrates balloons 104C, 104B, and 104C in an inflated state, according to some examples.

FIG. 10A illustrates a side view of a balloon catheter system 100 with a stent-like structure 110 positioned over balloons 104A, 104B, and 104C in an uninflated state, according to some examples.

FIG. 10B illustrates a side view of the balloon catheter system 100 in which the balloons 104A, 104B, and 104C are in various states of inflation, depending on how the operator actuates inflation, according to some examples.

FIGS. 11A and 11B illustrate a side view of the balloon catheter system 100 deploying a stent-like structure 110 (e.g., a replacement valve framework) within a heart valve 10, according to some examples.

FIGS. 12A and 12B illustrate side views of the balloon catheter system 100 deploying a stent-like structure 110 within an aortic aneurysm 20, according to some examples.

FIG. 13 illustrates a side view of the balloon catheter system 100 deploying a stent-like structure 110 within a thoracic aortic aneurysm 30, according to some examples.

FIG. 14 illustrates a side view of the balloon catheter system 100 deploying a stent-like structure 110 within a vessel containing plaque 40, according to some examples.

FIG. 15 illustrates a side view of a catheter 200 comprising mesh structures 204A, 204B, and 204C that may be selectively and individually expanded in diameter, according to some examples.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Typically, balloon catheters comprise a single balloon on the end of a catheter body that inflates to a generally cylindrical shape with a mostly uniform diameter. These balloon catheters can be used for delivering stents, artificial valves, or similar devices, as well as expanding areas within a patient, such as for angioplasty or valvuloplasty.

However, some areas of use within a patient may have an irregular shape. For example, locations near an aneurysm, near a large plaque deposit, or within an annulus of a valve often include shapes that vary in diameter and/or have non-symmetrical shapes. These uniformity variations can increase the difficulty of adequately expanding a device or a target portion of anatomy.

Aspects of a catheter are described in this specification which may improve delivery, expansion, or other therapy at areas having irregular shapes, variations in diameter, or both. The catheter may include a plurality of independently expandable expansile elements. Expansile elements may include balloons, mesh structures, strut frameworks, hydrogel, or similar structures.

The catheter may also include an expansion mechanism that, when adjusted by a user to an actuated state, selectively expands some or all of the expansile elements at various times and to different various diameters. Expansion mechanisms may include one or more expansion mechanism members, including a longitudinally movable inflation tube that may individually direct inflation media into individual balloons, elongated control wires that are longitudinally movable to expand mesh expansile members, longitudinally movable outer sheaths that selectively expose individual self-expanding expansile members, electrically activated hydrogel that selectively expands when current is directed to it, or similar variations.

An example balloon catheter may include a plurality of independently inflatable balloons or a plurality of independently inflatable chambers of a single balloon. Each of these balloons/chambers may expand to a different desired diameter to accommodate any irregular shapes or diameters of the target anatomy. Additionally or alternatively, each of these balloons/chambers may be individually inflated in a desired sequence which may be further helpful during certain procedures.

For simplicity, the terms “balloon” or “balloons” may be used in this specification. However, these terms should be understood to include discrete, physically separate balloons, physically touching or connected balloons, or a single balloon structure with multiple inflation chambers. Further, these balloons may have balloon chambers that are completely isolated from each other, partially in communication with each other, or only selectively/periodically in communication with each other.

In some examples, a distal region of the balloon catheter may include two or more balloons that can be independently inflated. Each balloon may be disposed over at least one catheter body opening or port which opens into an interior body passage extending from the distal region to a proximal end of the catheter body. Inflation media is directed to each of the catheter body ports by an elongated inflation tube. The inflation tube is movable (e.g., longitudinally slidable and/or rotatable) within the interior body passage of the catheter body and may be optionally removable from the catheter body.

A distal portion of the inflation tube may have an inflation port (e.g., on its side) which may be longitudinally and rotationally aligned with one of the ports of the catheter body into one of the plurality of balloons. When inflation media is injected into a proximal end of the inflation tube, the chamber of the balloon the inflation tube is aligned with inflates. The inflation tube may be longitudinally moved and aligned with other ports of the catheter body so that other balloons may be subsequently inflated.

Specific example uses of the balloon catheters of this specification may include stent delivery on a thoracic aortic aneurysm, stent delivery for an abdominal aortic aneurysm, trans aortic valve repair or replacement, superior vena cava stenosis, angioplasty, valvuloplasty, and similar treatments.

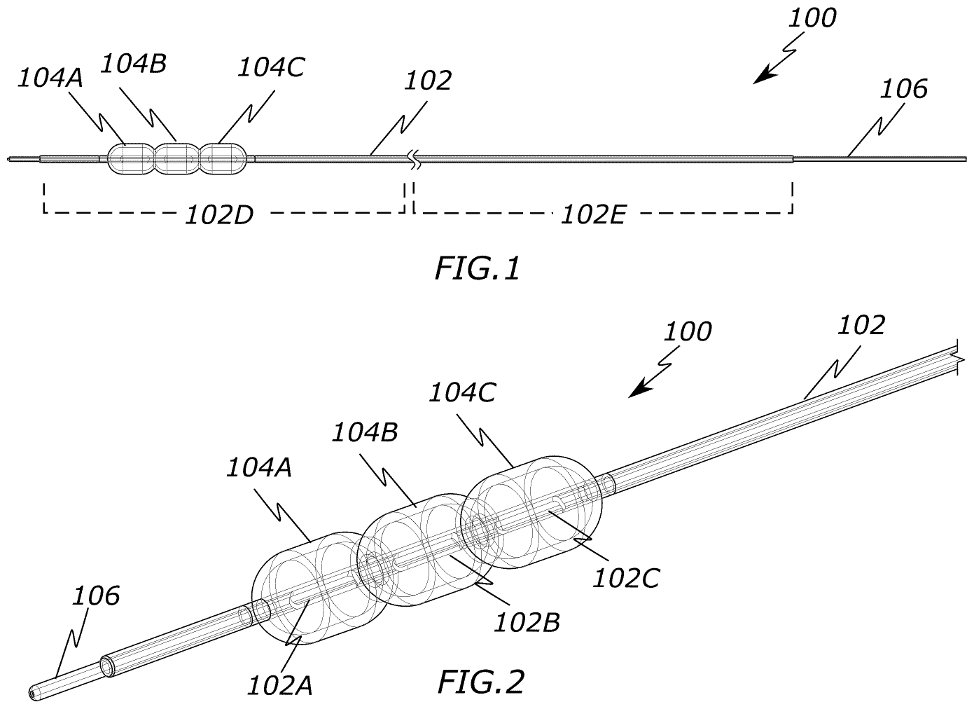

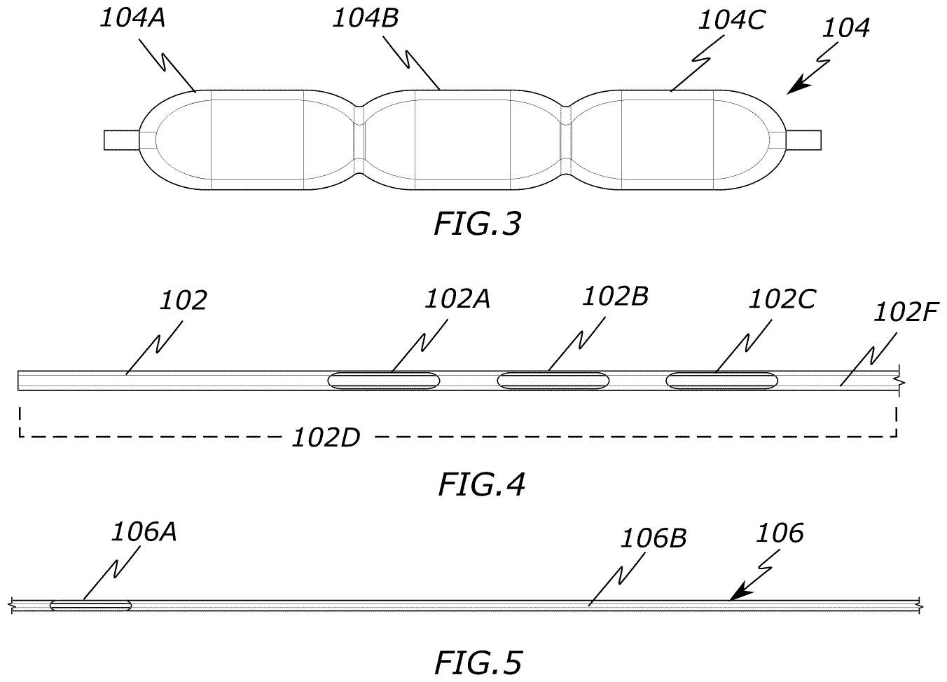

FIG. 1 illustrates a side view of an example balloon catheter system 100 with a plurality of balloons 104A, 104B, and 104C located within a distal region 102D of a catheter body 102 that may be separately and independently inflated relative to each other. FIG. 2 illustrates a perspective view of a portion of the distal region 102D of the catheter body 102. FIG. 3 illustrates a side view of a balloon assembly 104 comprising the plurality of balloons 104A, 104B, and 104C. FIG. 4 illustrates a side view of the distal region 102D of the catheter body 102. FIG. 5 illustrates a side view of the inflation tube 106. These figures will be discussed concurrently below.

As best illustrated in FIGS. 1 and 4, the balloon catheter system 100 may include an elongated catheter body 102. The catheter body 102 may include at least one interior body passage 102F that extends from an opening at a proximal end of a proximal region 102E (FIG. 1) of the catheter body 102 (e.g., a catheter hub) to a distal region 102D (FIG. 2, 4) of the catheter body 102. The distal region 102D of the catheter body may have a variety of different lengths, depending on the use, target location, and the approach to the target location. However, in one example, the distal region 102D of the catheter body 102 may have a length from a distal end of the catheter body 102 within an inclusive range of 0 mm to 50 mm. In another example, the distal region 102D of the catheter body 102 may have a length from the distal end of the catheter body 102 within an inclusive range of 10 mm to 40 mm.

As further illustrated in FIG. 4, the catheter body 102 also includes a plurality of catheter body ports 102A, 102B, and 102C that open into the at least one interior body passage 102F of the catheter body 102. The catheter body ports 102A, 102B, 102C form openings or passages through the wall of the catheter body 102 to connect the interior body passage 102F with the interior chambers of a respective balloon 104A, 104B, and 104C. The catheter body ports 102A, 102B, 102C are longitudinally spaced apart from each other and may be generally located within the distal region of the catheter body 102 or near a distal end of the catheter body 102. In the illustrated example, the catheter body ports 102A, 102B, and 102C are illustrated as being located generally at the same radial or circumferential position relative to each other, however, the ports 102A, 102B, and 102C may also be located at different radial or circumferential positions relative to each other.

Referring to FIG. 2, each balloon 104A, 104B, and 104C is located over a corresponding catheter body port 102A, 102B, 102C so that each catheter body port may open into only one of the balloons (e.g., there is a one-to-one relationship such that each catheter body port is in fluid communication with only one balloon). Aside from the catheter body ports 102A, 102B, 102C, the chamber within each balloon may be sealed from an outside atmosphere, as well as sealed from adjacent balloons 104A, 104B, and 104C. Alternatively, the chambers of each balloon may have a relatively small passage into adjacent balloon chambers.

The example balloon catheter system 100 is illustrated with three balloons in these figures, however, two or more balloons may alternatively be included (e.g., 2, 3, 4, 5, 6, 7, 8, 9, or 10). Each of the plurality of balloons 104A, 104B, 104C may be the same length as each other or have different lengths (e.g., lengths within an inclusive range of about 2 mm to 20 mm each). Each of the balloons may be physically touching or directly connected to each other, or may be longitudinally spaced apart or physically separated from each other.

An inflation tube 106 may be used to inflate each of the balloons 104A, 104B, and 104C. As seen in FIG. 5, the inflation tube 106 may be an elongated tube with an internal inflation passage 106B extending between a proximal region and a distal region. At the proximal region, the internal inflation passage is in communication with an inflation device (e.g., a syringe). At the distal region, the internal inflation passage 106B is in communication with an inflation opening 106A (e.g., distal inflation port). The inflation opening 106A may be located along a side or through a sidewall of the inflation tube 106 and may have a size smaller, larger, or about the same size as one of the catheter body ports 102A, 102B, 102C.

The inflation tube 106 may be positioned within the interior body passage 102F of the catheter body 102 and the inflation opening 106A may be longitudinally and rotationally positioned to align with one of the catheter body ports 102A, 102B, 102C. The inflation tube 106 may have a length such that a proximal end of the inflation tube 106 is positioned proximally outside of the catheter body 102 during use, allowing a user to grasp and move the inflation tube 106 relative to the catheter body 102. When media is injected into the internal inflation passage 106B of the inflation tube 106, the media passes through the inflation opening 106A. If the inflation opening 106A is positioned under or aligned with one of the catheter body ports 102A, 102B, 102C, the media passes through one of those aligned catheter body ports 102A, 102B, 102C and into the chamber of one of the corresponding balloons 104A, 104B, or 104C.

Optionally the inflation tube 106 and the interior body passage of the catheter body 102 may include a feature that maintains only a specific rotational orientation between the inflation tube 106 and catheter body 102 so that the inflation opening 106A always maintains a rotational orientation that aligns with the catheter body ports 102A, 102B, 102C. For example, the inflation tube 106 and/or the catheter body 102 may include mating grooves, ridges, diameter shapes, and similar features may lock rotational orientation of the inflation tube 106 relative to the catheter body 102.

One or more seals or gaskets may also be included in the inflation tube 106 and/or the catheter body 102 to prevent unwanted leakage of the inflation media. For example, areas surrounding the catheter body ports 102A, 102B, 102C (e.g., within the interior body passage of the catheter body 102) may include seals/gaskets, and/or an area on the outer surface of the inflation tube 106 surrounding the inflation opening 106A may also include a seal/gasket.

The balloon catheter system 100 may also include one or more features that indicate a position of the inflation opening 106A and/or the catheter body ports 102A, 102B, 102C to that the user may move and align the inflation opening 106A as needed to inflate a desired balloon 104A, 104B, 104C.

In one example, one or more radiopaque markers may be positioned immediately proximally, immediately distally, and/or circumferentially relative to the inflation opening 106A, allowing the location of the inflation opening 106A to be visualized during a procedure. Additionally or alternatively, radiopaque markers may be positioned immediately proximally, immediately distally, and/or circumferentially relative to each of the catheter body ports 102A, 102B, 102C, allowing the location of these ports to be visualized during a procedure.

Additionally or alternatively, the proximal portion of the inflation tube 106 may include a plurality of visual markers (e.g., ink, engraving, etc.) that are spaced to align with a proximal end of the catheter body 102 when the inflation opening 106A is at a predetermined position (e.g., longitudinally aligned with a catheter body port 102A, 102B, 102C). Hence, the inflation tube 106 may have a first proximal marker indicating alignment with the catheter body port 102A, a second proximal marker indicating alignment with the catheter body port 102B, and a third proximal marker indicating alignment with the catheter body port 102C.

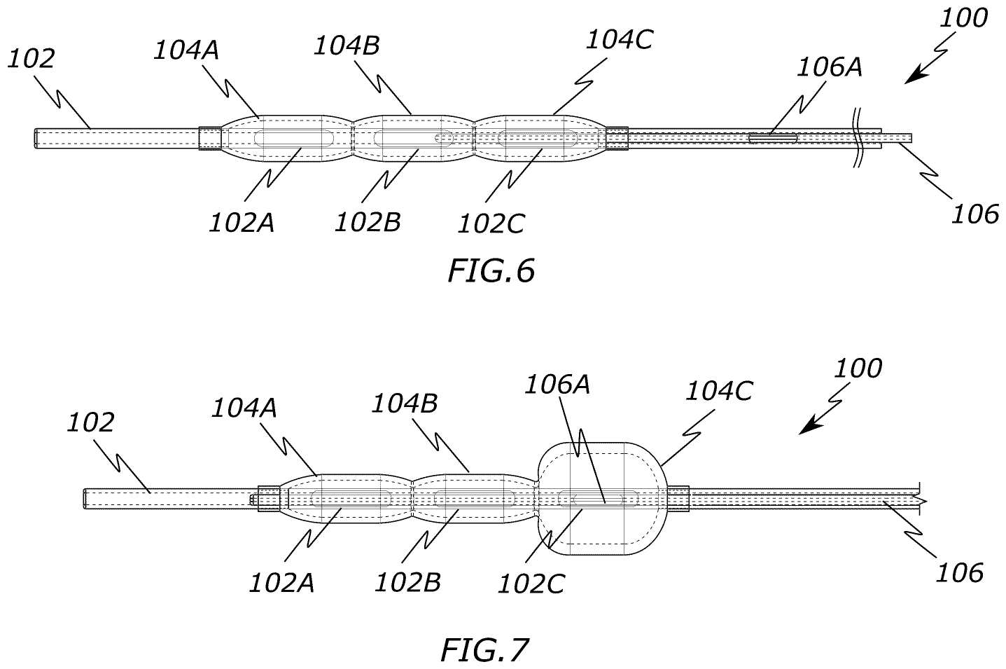

FIGS. 6-9 illustrate a side view of the balloon catheter system 100 in various inflation states or configurations and also an example method of inflating the balloon catheter system 100, as discussed further below.

FIG. 6 illustrates balloons 104A, 104B, and 104C in a deflated state. As shown in FIG. 6, the inflation opening 106A is positioned proximally of catheter body ports 102A, 102B, 102C. In such a configuration where the inflation opening 106A is not aligned with catheter body ports 102A, 102B, or 102C, if inflation media is injected into the internal inflation passage 106B of the inflation tube 106, balloons 104A, 104B, and 104C may maintain a deflated state because inflation fluid is unable to enter within them. In other words, the inflation opening 106A is sealed off from the catheter body ports 102A, 102B, and 102C when not respectively aligned.

However, some positions of the inflation opening 106A within the interior body passage of the catheter body 102 may allow for all of the balloons 104A, 104B, and 104C to be simultaneously inflated. In some examples, this may be achieved by including regions of the interior body passage 102F with two different diameters. A first region of the interior body passage 102F may have a diameter that closely fits or is about the same size as the outer diameter of the inflation tube 106. In that respect, the inflation opening 106A may remain blocked when positioned in this closely fitting first diameter region since the inflation opening 106A is located on a side of the inflation tube 106.

A second region of the interior body passage 102F may have a diameter that is larger than the first region and provides space (e.g., a gap) between the interior body passage 102F and the outer surface of the inflation tube 106. When the inflation opening 106A is positioned within this larger diameter second region, the inflation opening 106A remains open to and in communication with the interior body passage 102F, allowing media to pass out of the inflation opening 106A between the gap between the interior body passage 102F and the inflation tube 106, down the interior body passage 102F, into each of the catheter body ports 102A, 102B, and 102C, and finally into the chambers of each of the balloons 104A, 104B, and 104C.

In one specific example seen in FIG. 1, the closely fitting first diameter region of the interior body passage 102F may be located along the distal region 102D of the catheter body 102. The larger diameter second region of the interior body passage 102F may be located along the proximal region 102E. In that respect, if the user positions the inflation opening 106A of the inflation tube 106 within the proximal region 102E, inflation media will pass distally into each of the catheter body ports 102A, 102B, and 102C, and finally into the chambers of each of the balloons 104A, 104B, and 104C. If the user positions the inflation opening 106A of the inflation tube 106 within the distal region 102D, the smaller diameter of the interior body passage 102F in that region either prevents any inflation media from escaping from the inflation tube 106 or only allows the inflation media to pass into one of the catheter body ports 102A, 102B, and 102C, and balloons 104A, 104B, and 104C.

FIG. 7 illustrates balloon 104C at an inflated state in response to injecting inflation media when the inflation opening 106A is aligned with catheter body port 102C. The inflation tube 106 may be distally advanced within the catheter body 102 from the position in FIG. 6 so that the inflation opening 106A aligns with catheter body port 102C. When inflation media is injected into the inflation passage of the inflation tube 106, the inflation media causes only the balloon 104C to inflate because the inflation opening 106A is aligned with catheter body port 102C.

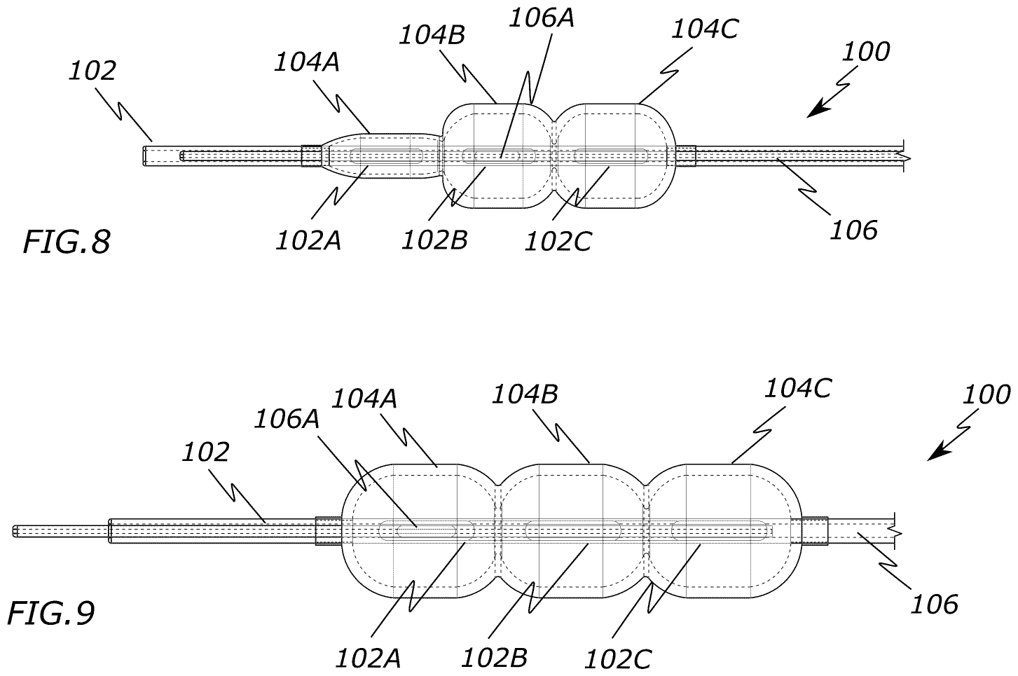

FIG. 8 illustrates balloons 104C and 104B in an inflated state. The inflation tube 106 may be further advanced from the position in FIG. 7 distally within the catheter body 102 so that the inflation opening 106A aligns with catheter body port 102B. When inflation media is injected into the inflation passage of the inflation tube 106, the inflation media causes only the balloon 104B to inflate because the inflation opening 106A is aligned with catheter body port 102B.

FIG. 9 illustrates balloons 104C, 104B, and 104C in an inflated state. The inflation tube 106 can be further advanced from the position in FIG. 8 distally within the catheter body 102 so that the inflation opening 106A aligns with catheter body port 102A. When inflation media is injected into the inflation passage of the inflation tube 106, the inflation media causes only the balloon 104A to inflate because the inflation opening 106A is aligned with catheter body port 102A.

An operator may choose to fill the balloons 104A, 104B, and 104C in any order. For example, the middle balloon 104B may be inflated first or last, the distal balloon 104A may be inflated first or last, or the proximal balloon 104C may be inflated first or last. This may be beneficial to an operator from an ease of use or patient safety perspective if the vasculature surrounding the balloons is not uniform (e.g., blocked, stenosis, abnormal anatomy, arterial lesion, etc.).

Additionally, an operator may choose to fill the balloons 104A, 104B, and 104C to different inflation diameters relative to each other. For example, the middle balloon 104B may be inflated larger or smaller than the adjacent balloons 104A and 104C. In another example the distal balloon 104A may be inflated larger or smaller than the balloons 104B and 104C. In another example the proximal balloon 104C may be inflated larger or smaller than the balloons 104A and 104B.

The balloons 104A, 104B, and 104C may be individually deflated in a similar manner as the inflation process, either partially or completely. For example, the inflation opening 106A of the inflation tube 106 may be moved to align with one of the catheter body ports 102A, 102B, and 102C. A proximal end of the inflation tube 106 may be adjusted to remove media from the internal inflation passage 106B, for example, by using a syringe to withdraw media or by opening a valve that releases the media.

If the interior body passage 102F with different diameter regions are present, all of the balloons 104A, 104B, and 104C may be simultaneously deflated by moving the inflation opening 106A to the proximal region 102E having the larger diameter which creates a pathway in communication with all of the interior chambers of the balloons 104A, 104B, and 104C. Hence, removing media from the inflation tube 106 (e.g., via syringe or valve) may simultaneously remove media from all of the balloons 104A, 104B, and 104C at once.

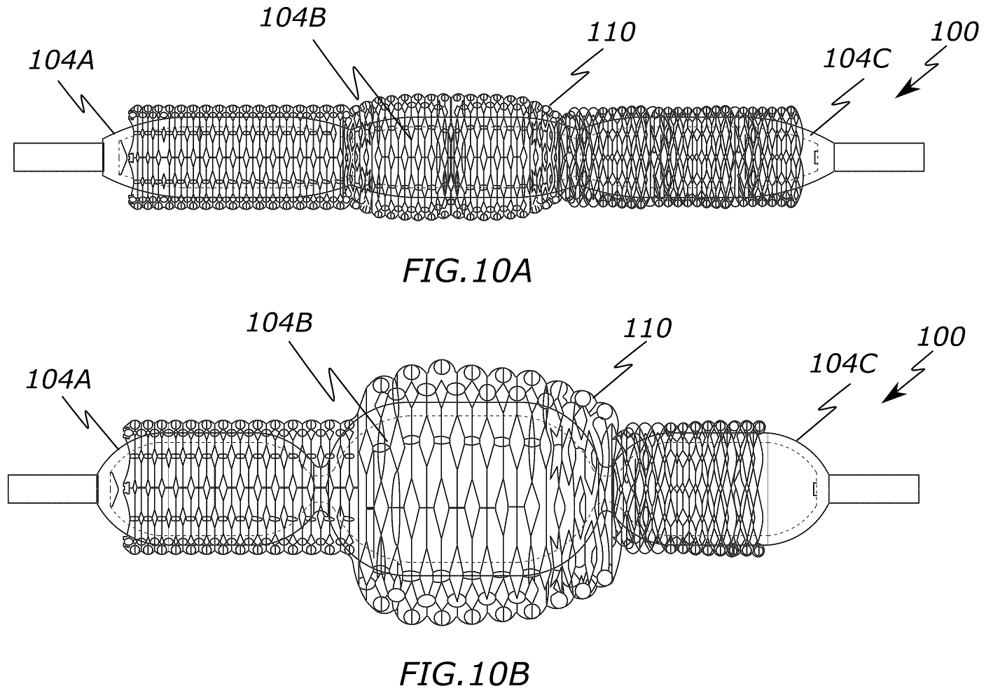

FIG. 10A illustrates a side view of a balloon catheter system 100 with a stent-like structure 110 positioned over balloons 104A, 104B, and 104C in an uninflated state. This uninflated state may be used when the balloon catheter system 100 is advanced to a desired location within a patient. Alternatively, the stent-like structure 110 may be first delivered to a target location within the patient and the balloons 104A, 104B, and 104C may be positioned within the lumen of the stent-like structure 110.

FIG. 10B illustrates a side view of the balloon catheter system 100 in which the balloons 104A, 104B, and 104C are in various states of inflation, depending on how the operator actuates inflation. In this example, the balloon 104B is inflated to a much larger diameter than the balloons 104A and 104C, at least initially. However, any combination of larger and smaller diameters, as previously disclosed, may be possible. Additionally, such combinations also apply if more or less than three balloons are included on the balloon catheter system 100.

Having the ability to choose different diameter sizes and sequences for the balloons 104A, 104B, and 104C may be beneficial to an operator from an ease of use or patient safety perspective if the vasculature surrounding the balloons is not uniform (e.g., blocked, stenosis, abnormal anatomy, arterial lesion, etc.). There may also be additional benefits, for example, a single pre-loaded stent-like structure 110 on a balloon (as described herein) may provide the operator with the option of selecting from two different methods of inflating the balloon(s) and expanding the stent-like structure 110, either individually (by inflating balloons 104A, 104B, and 104C sequentially, individually, and/or independently) or as a group (simultaneous inflation of all balloons). This may benefit the operator by providing a single intravascular device that can accommodate various anatomies and/or disease states, and if unexpected anatomies and disease states are encountered during a procedure, the operator may be able to expand the balloon(s) and/or deploy the stent-like structure without having to remove the device from the patient. This one-size-fits-all type approach may provide reduced operation times, reduced waste, improved patient outcomes, and cost savings. Additional benefits may include improved deployment accuracy of the stent-like structure 110, improved anchoring (and/or initial anchoring) of the stent-like structure 110 to the targeted vessel wall, and improved wall apposition of the stent (and corresponding improvements in patient outcomes related to aneurysm occlusion).

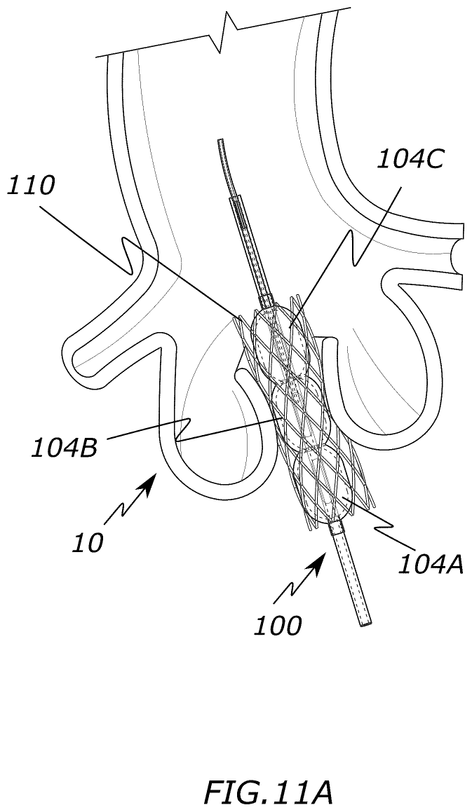

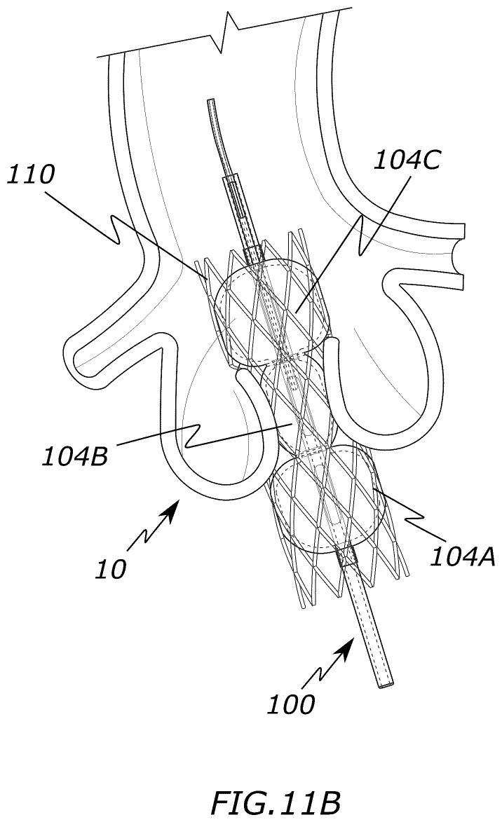

FIGS. 11A and 11B illustrate a side view of the balloon catheter system 100 deploying a stent-like structure 110 (e.g., a replacement valve framework) within a heart valve 10.

In FIG. 11A, the balloon catheter system 100 and stent-like structure 110 are positioned between leaflets of the heart valve 10.

In FIG. 11B, the balloons are inflated such that balloons 104A and 104C are greatly increased in diameter on each side of the leaflets relative to the middle balloon 104B. Since many valves tend to have a somewhat hourglass shape, the stent-like structure 110 may be expanded in a way that better conforms to the underlying structure of the heart valve 10. specifically, the balloons 104A and 104C can better expand the stent-like structure 110 against the upper and lower shapes of the heart valve 10, while the middle balloon 104B can better accommodate the leaflets and annulus of the heart valve 10. Again, depending on the desired positioning of the balloon catheter system 100 in the heart valve 10 and the shape of the heart valve 10, other inflations profiles and sequences may be desirable.

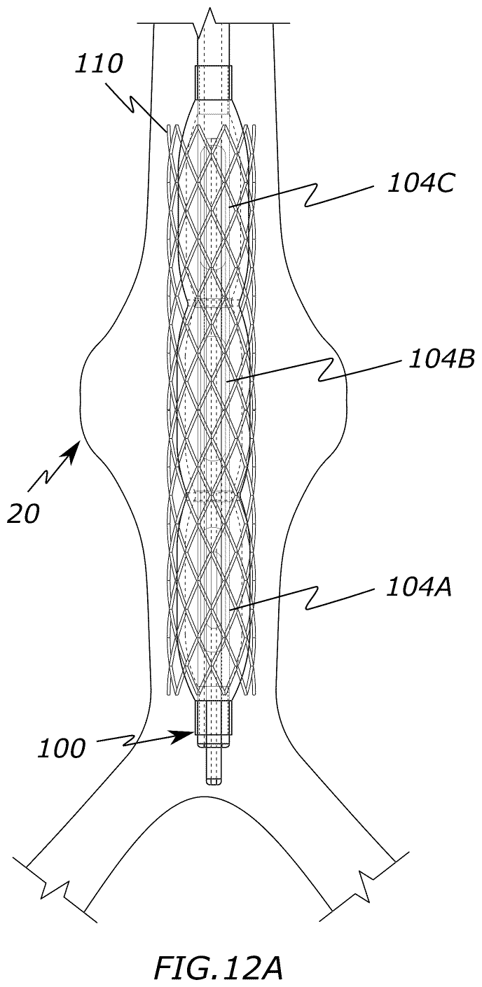

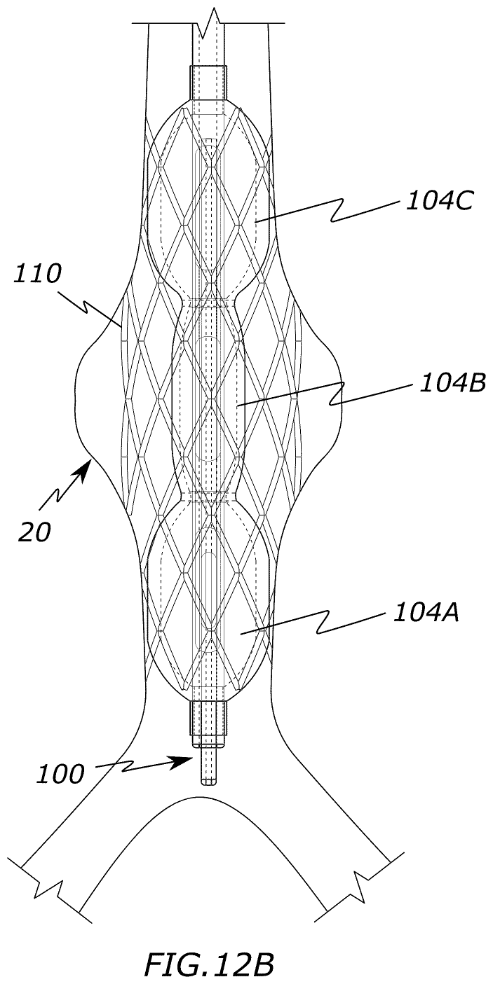

FIGS. 12A and 12B illustrate side views of the balloon catheter system 100 deploying a stent-like structure 110 within an aortic aneurysm 20.

In FIG. 12A, the balloon catheter system 100 is positioned through the aortic aneurysm 20 so that the end balloons 104A and 104C are located beyond each side of the aortic aneurysm 20.

In FIG. 12B, the middle balloon 104B may be inflated in size first to expand a middle of the stent-like structure 110 to a desired diameter and then deflated, followed by inflating the end balloons 104A and 104C to expand and anchor the stent-like structure 110. Such sequential inflation, in addition to differential inflation sizes, may be helpful in various circumstances for a variety of purposes, such as improving anchoring force of the stent-like structure 110, improving the expansion location of the stent-like structure 110 (e.g., if it foreshortens during expansion), or reducing stress on portions of a vessel wall.

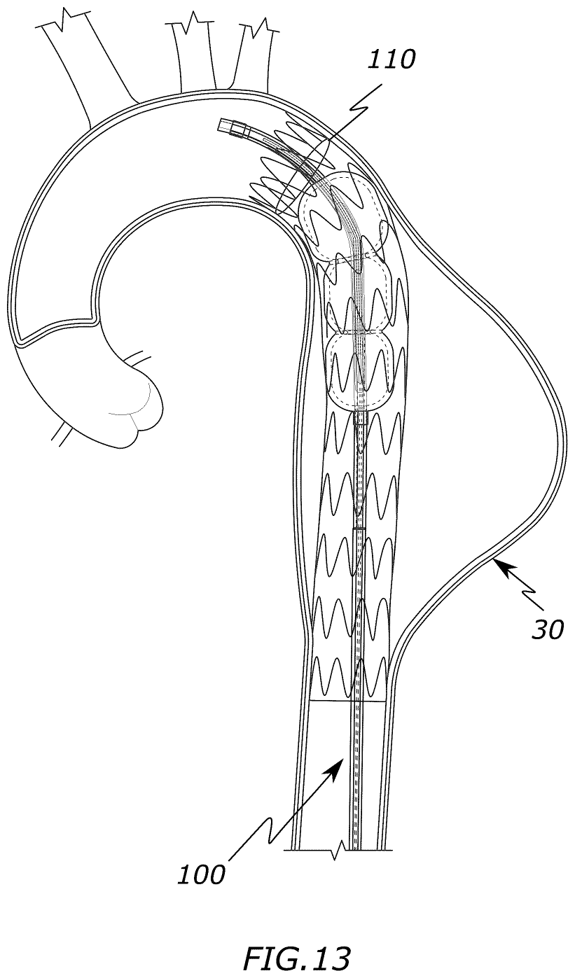

FIG. 13 illustrates a side view of the balloon catheter system 100 deploying a stent-like structure 110 within a thoracic aortic aneurysm 30. Note, the catheter system 100 is illustrated such that the length of the array of the balloons does not extend the entire length of the stent-like structure 110 and may be advantageous in certain circumstances, such as for incremental expansion of a relatively long stent-like structure 110. However, the array of balloons may alternatively extend the length of the stent-like structure 110 or even beyond the length of the stent-like structure 110 in the FIG. 13 example. These length variations are applicable to any other examples or embodiments of the balloon catheter system 100 described in this specification.

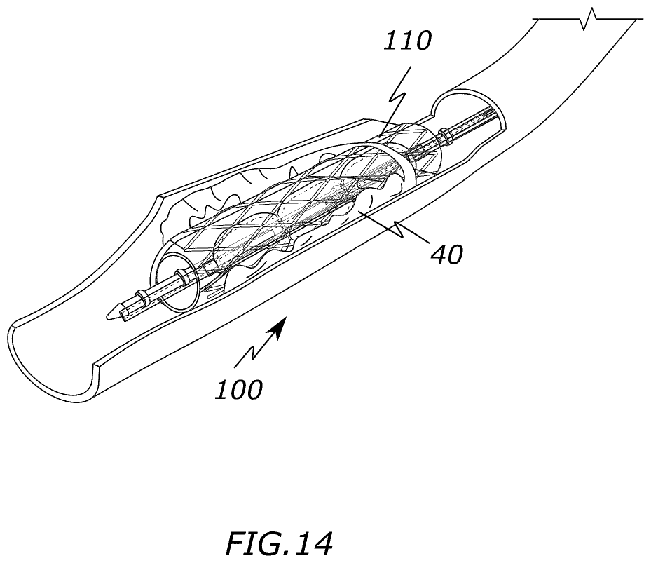

FIG. 14 illustrates a side view of the balloon catheter system 100 deploying a stent-like structure 110 within a vessel containing plaque 40. Depending on the shape of the plaque 40, inflation of the balloons to different diameters may be helpful for expanding the vessel and securing the stent-like structure 110. For example, the expansion profile of FIGS. 10A and 10B may be helpful to help compress the plaque 40 and open diameter of the vessel.

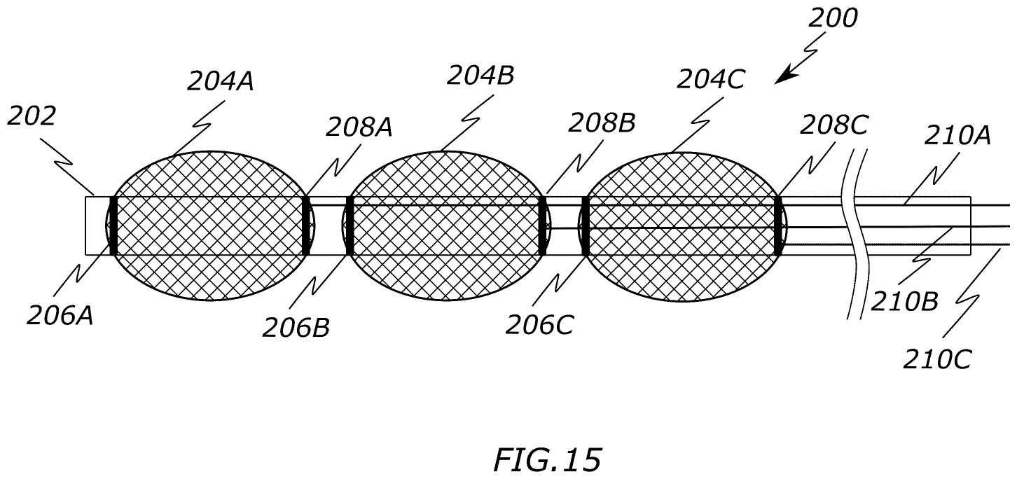

In some examples, the expansile element may include an expandable mesh structure. For example, FIG. 15 illustrates a side view of a catheter 200 comprising mesh structures 204A, 204B, and 204C that may be selectively and individually expanded in diameter.

In some examples, the mesh structures 204A, 204B, and 204C may comprise a tubular mesh shape that is located around the catheter body 202. One end of each of the mesh structures 204A, 204B, and 204C may be fixed in place relative to the catheter body 202 while the other may be longitudinally slidable relative to the catheter body 202. For example, distal mounting fixtures 206A, 206B, and 206C may fix the distal ends of the mesh structures 204A, 204B, and 204C to the catheter body 202, respectively. Proximal mounting fixtures 208A, 208B, and 208C may be fixed to proximal ends of each of the mesh structures 204A, 204B, and 204C respectively and may further be slidably connected to the catheter body 202. For example, the proximal mounting fixtures 208A, 208B, and 208C may be a ring on the outside of the catheter body 202 or on the inside of the catheter body 202.

Each of the proximal mounting fixtures 208A, 208B, and 208C may also be connected to control wires 210A, 210B, and 210C respectively which allow a user to adjust the longitudinal position of each of the proximal mounting fixtures 208A, 208B, and 208C along the catheter body 202. Each of the control wires 210A, 210B, and 210C may be located within the catheter body 202 or along an outer surface of the catheter body 202. Each of the control wires 210A, 210B, and 210C may extend proximally out of the proximal end of the catheter body 202 so that the user may grasp them directly or they may further connect to a handle mechanism that longitudinally moves the control wires 210A, 210B, and 210C (e.g., via individual sliders on the handle).

As a user distally advances one of the control wires 210A, 210B, or 210C, a respective proximal mounting fixture 208A, 208B, or 208C is also moved distally causing the respective mesh structure 204A, 204B, or 204C to radially expand to a desired diameter/size. Conversely, as a user proximally retracts one of the control wires 210A, 210B, or 210C, a respective proximal mounting fixture 208A, 208B, or 208C is also moved proximally causing the respective mesh structure 204A, 204B, or 204C to radially contract to a desired diameter/size. As with the other examples discussed in this specification, the catheter 200 may be used to expand a stent, angioplasty, valvuloplasty, or other uses.

### Claim Bank

Clause 1. An expandable catheter system, comprising: a catheter body; a plurality of expansile elements connected at a distal region of the catheter body; and, an expansion mechanism having an actuated state that independently adjusts a radial size of each of the plurality of expansile elements.

Clause 2. The expandable catheter system of clause 1, wherein the plurality of expansile elements include balloons and wherein the expansion mechanism includes an inflation tube longitudinally movable within the catheter body between at least a first position in communication with a first balloon of the plurality of balloons, and a second position in communication with a second balloon of the plurality of balloons.

Clause 3. The expandable catheter system of clause 2, wherein the catheter body comprises an interior body passage extending between a proximal region and a distal region of the catheter body; and wherein the inflation tube is longitudinally movable within the interior body passage between the at least first position and second position.

Clause 4. The expandable catheter system of clause 3, wherein of all of the plurality of balloons, the inflation tube is only in communication with the first balloon when in the first position.

Clause 5. The expandable catheter system of clause 4, wherein of all of the plurality of balloons, the inflation tube is only in communication with the second balloon when in the second position.

Clause 6. The expandable catheter system of clause 3, wherein of all of the plurality of balloons, the inflation tube is only in communication with the first balloon when in the first position and wherein the inflation tube is only in communication with the first balloon and the second balloon when in the second position.

Clause 7. The expandable catheter system of clause 2, wherein the first balloon and the second balloon each include inflation chambers that are completely isolated from each other, partially in communication with each other, or selectively in communication with each other.

Clause 8. The expandable catheter system of clause 3, wherein the catheter body comprises a first catheter body port located underneath the first balloon and a second catheter body port located underneath the second balloon.

Clause 9. The expandable catheter system of clause 8, wherein the inflation tube further comprises an inflation port opening through a sidewall of the inflation tube and into an inflation passage of the inflation tube.

Clause 10. The expandable catheter system of clause 9, further comprising rotational orientation lock that maintains a rotational orientation of the inflation tube relative to the catheter body.

Clause 11. The expandable catheter system of clause 9, further comprising one or more seals located around 1) the inflation port opening and/or located around 2) the first catheter body port and the second catheter body port.

Clause 12. The expandable catheter system of clause 2, further comprising a stent positioned over the plurality of balloons.

Clause 13. The expandable catheter system of clause 2, further comprising a replacement valve framework positioned over the plurality of balloons.

Clause 14. The expandable catheter system of clause 2, wherein at least some of the plurality of balloons have inflated diameters that are different from each other.

Clause 15. The expandable catheter system of clause 2, wherein the plurality of balloons comprises the first balloon, the second balloon, and a third balloon, and wherein the second balloon is positioned between the first balloon and the third balloon, and wherein an inflated diameter of the second balloon is larger or smaller than inflated diameters of the first balloon and the third balloon.

Clause 16. The expandable catheter system of clause 2, wherein the plurality of balloons comprises the first balloon, the second balloon, and a third balloon, and wherein the inflation tube is moved between the first position, the second position, and a third position to sequentially inflate the first balloon first, the second balloon second, and the third balloon third.

Clause 17. The expandable catheter system of clause 2, wherein the inflation tube is movable to a third position to remove inflation fluid from the plurality of balloons.

Clause 18. The expandable catheter system of clause 1, wherein the plurality of expansile elements include a plurality of mesh structures and wherein the expansion mechanism includes one or more control wires connected to the plurality of mesh structures.

Clause 19. A balloon catheter system, comprising: a catheter body comprising an interior body passage extending between a proximal region and a distal region of the catheter body; a plurality of balloons connected at the distal region of the catheter body; wherein each balloon is separately in communication with the interior body passage; and, an inflation tube comprising a distal inflation opening; wherein the inflation tube is longitudinally movable between at least a first position in communication with a first balloon of the plurality of balloons, and a second position in communication with a second balloon of the plurality of balloons.

Clause 20. A balloon catheter system, comprising: a catheter body; a plurality of balloons connected at a distal region of the catheter body; and, an inflation tube means for longitudinally moving between positions within the catheter body and establishing an inflation passage between only one of the plurality of balloons at a time.

Clause 21. A method of using a balloon catheter system, comprising: longitudinally aligning a distal inflation opening of an inflation tube with a first port of an interior body passage of a catheter body; inflating a first balloon in communication with the first port; longitudinally aligning the distal inflation opening of the inflation tube with a second port of an interior body passage of the catheter body; and, inflating a second balloon in communication with the second port.

Clause 22. The method of clause 21, further comprising longitudinally aligning the distal inflation opening of the inflation tube with a third port of the interior body passage of the catheter body, and inflating a third balloon in communication with the third port.

What is claimed is:

1. An expandable catheter system, comprising:
a catheter body;
a plurality of expansile elements connected at a distal region of the catheter body; and,
an expansion mechanism having an actuated state that independently adjusts a radial size of each of the plurality of expansile elements.

2. The expandable catheter system of claim 1, wherein the plurality of expansile elements include a plurality of balloons and wherein the expansion mechanism includes an inflation tube longitudinally movable within the catheter body between at least a first position in communication with a first balloon of the plurality of balloons, and a second position in communication with a second balloon of the plurality of balloons.

3. The expandable catheter system of claim 2, wherein the catheter body comprises an interior body passage extending between a proximal region and a distal region of the catheter body; and wherein the inflation tube is longitudinally movable within the interior body passage between the at least first position and second position.

4. The expandable catheter system of claim 3, wherein of all of the plurality of balloons, the inflation tube is only in communication with the first balloon when in the first position.

5. The expandable catheter system of claim 4, wherein of all of the plurality of balloons, the inflation tube is only in communication with the second balloon when in the second position.

6. The expandable catheter system of claim 3, wherein of all of the plurality of balloons, the inflation tube is only in communication with the first balloon when in the first position and wherein the inflation tube is only in communication with the first balloon and the second balloon when in the second position.

7. The expandable catheter system of claim 2, wherein the first balloon and the second balloon each include inflation chambers that are completely isolated from each other, partially in communication with each other, or selectively in communication with each other.

8. The expandable catheter system of claim 3, wherein the catheter body comprises a first catheter body port located underneath the first balloon and a second catheter body port located underneath the second balloon.

9. The expandable catheter system of claim 8, wherein the inflation tube further comprises an inflation port opening through a sidewall of the inflation tube and into an inflation passage of the inflation tube.

10. The expandable catheter system of claim 9, further comprising rotational orientation lock that maintains a rotational orientation of the inflation tube relative to the catheter body.

11. The expandable catheter system of claim 9, further comprising one or more seals located around 1) the inflation port opening and/or located around 2) the first catheter body port and the second catheter body port.

12. The expandable catheter system of claim 2, further comprising a stent positioned over the plurality of balloons.

13. The expandable catheter system of claim 2, further comprising a replacement valve framework positioned over the plurality of balloons.

14. The expandable catheter system of claim 2, wherein at least some of the plurality of balloons have inflated diameters that are different from each other.

15. The expandable catheter system of claim 2, wherein the plurality of balloons comprises the first balloon, the second balloon, and a third balloon, and wherein the second balloon is positioned between the first balloon and the third balloon, and wherein an inflated diameter of the second balloon is larger or smaller than inflated diameters of the first balloon and the third balloon.

16. The expandable catheter system of claim 2, wherein the plurality of balloons comprises the first balloon, the second balloon, and a third balloon, and wherein the inflation tube is moved between the first position, the second position, and a third position to sequentially inflate the first balloon first, the second balloon second, and the third balloon third.

17. The expandable catheter system of claim 2, wherein the inflation tube is movable to a third position to remove inflation fluid from the plurality of balloons.

18. The expandable catheter system of claim 1, wherein the plurality of expansile elements include a plurality of mesh structures and wherein the expansion mechanism includes one or more control wires connected to the plurality of mesh structures.

19. A balloon catheter system, comprising:
a catheter body comprising an interior body passage extending between a proximal region and a distal region of the catheter body;
a plurality of balloons connected at the distal region of the catheter body; wherein each balloon is separately in communication with the interior body passage; and,
an inflation tube comprising a distal inflation opening; wherein the inflation tube is longitudinally movable between at least a first position in communication with a first balloon of the plurality of balloons, and a second position in communication with a second balloon of the plurality of balloons.

20. A balloon catheter system, comprising:
a catheter body;
a plurality of balloons connected at a distal region of the catheter body; and,
an inflation tube means for longitudinally moving between positions within the catheter body and establishing an inflation passage between only one of the plurality of balloons at a time.
